(19) United States
(12) Patent Application Publication
(10) Pub. No.: US 2014/0012174 A1
(43) Pub. Date: Jan. 9, 2014

(54) Adhesive Bandage

(71) Applicants: Mao-Tsun Wu, Taichung City (TW); Yen-Ni Hung, Taipei City (TW); Chen Yang, Guishan Township (TW); Hui-Ping Liu, Taipei City (TW)

(72) Inventors: Mao-Tsun Wu, Taichung City (TW); Yen-Ni Hung, Taipei City (TW); Chen Yang, Guishan Township (TW); Hui-Ping Liu, Taipei City (TW)

(21) Appl. No.: 13/540,659

(22) Filed: Jul. 3, 2012

(51) Int. Cl. A61F 13/02

(52) U.S. Cl. 602/53

(57) ABSTRACT

An adhesive bandage is provided with a support cup having an annular marking formed on a periphery of an outer surface; a circular cushion member fitted in the support cup; a sliding cup; a sliding disc in the sliding cup and engaged the cushion member, the sliding disc being capable of sliding into the support cup; and a resilient main body including a transparent dome for concealing the sliding cup, the cushion member, the sliding disc, and the support cup, a first adhesive strip extending from a periphery of the dome; a second adhesive strip, opposite the first adhesive strip, extending from the periphery of the dome; a hook and loop fabric fastener on ends of the first and second adhesive strips.

### BACKGROUND OF THE INVENTION

1. Field of the Invention

The invention relates to prepared adhesive bandages for surgical dressings and more particularly to an adhesive bandage having an arrangement to allow a medical employee to clearly see the change of pressure applied thereto by observing a relative movement of a marking and a bottom edge of a sliding cup so as to adjust tightness of the adhesive bandage.

2. Description of Related Art

In clinical practice, doctors personally assess patients in order to diagnose, treat, and prevent disease using clinical judgment. After examination for signs and interviewing for symptoms, a doctor may order a blood test or other therapies. A blood test is a laboratory analysis performed on a blood sample that is usually extracted from a vein in the arm using a needle (i.e., venipuncture), or via fingerprick. In either case, how to stop bleeding is an important issue to address after drawing blood from a vein.

Regarding stopping bleeding after venipuncture, compression bandage is typically applied. However, it may not help anything improve but it can aggravate an already poor arterial supply. So a typical practice is that applying padding (e.g., absorbent cotton) to the wound, then applying a splint, and finally applying a compression bandage to hold the splint in place for protection.

For patients taking a blood test, adhesive bandage is typically applied. A typical adhesive bandage comprises a disc shaped pad (served as force applying point) and an adhesive strip extending from either end of the pad. The pad is pressed upon a punctured point. Next, the adhesive strips are affixed to the skin. While it is convenient, its attachment to the skin is not secure and pressure applied to the skin cannot be controlled precisely. In fact, its success depends totally on the experience of a medical practitioner. Moreover, pressure applied to the blood vessel varies depending upon the types of the blood vessels. For example, pressure applied to the arteries for stopping bleeding is greater than that applied to the arteries for contraction. For patients suffering veins or arteries diseases, pressure applied by the adhesive bandage is relatively low. Otherwise, thrombus may occur in the veins, the arteries, and/or a combination thereof.

Thus, the need for improvement still exists.

### SUMMARY OF THE INVENTION

It is therefore one object of the invention to provide an adhesive bandage comprising a support cup comprising a space defined therein, and an annular marking formed on a periphery of an outer surface; a circular cushion member fitted in the space of the support cup; a sliding cup comprising a space defined therein; a sliding disc disposed in the space of the sliding cup and engaged the cushion member, the sliding disc being capable of sliding into the space of the support cup and including an activation member formed on one surface and a guide member formed on a periphery; and a main body formed of silicone rubber and comprising a transparent dome for concealing the sliding cup, the cushion member, the sliding disc, and the support cup, a first adhesive strip extending from a periphery of the dome; a second adhesive strip, opposite the first adhesive strip, extending from the periphery of the dome; a hook and loop fabric fastener formed on ends of the first and second adhesive strips wherein the hook and loop fabric fastener is capable of being pressed together for releasable fastening; wherein the sliding disc compresses the cushion member in response to a sliding movement of the sliding cup with respect to the support cup so that a change of pressure applied to the adhesive bandage is visually brought about by a relative movement of the marking and a bottom edge of the sliding cup.

The above and other objects, features and advantages of the invention will become apparent from the following detailed description taken with the accompanying drawings.

### BRIEF DESCRIPTION OF THE DRAWINGS

FIG. 1 is a perspective view of an adhesive bandage according to a first preferred embodiment of the invention;

FIG. 2 is an exploded view of the pad;

FIG. 3 is a longitudinal sectional view of the assembled pad of FIG. 2 but viewed in upside down;

FIG. 4 is a longitudinal section view of the adhesive bandage affixed to the skin;

FIG. 5 is an environmental view of the adhesive bandage affixed to the arm of a patient; and

FIG. 6 is a perspective view of an adhesive bandage according to a second preferred embodiment of the invention.

### DETAILED DESCRIPTION OF THE INVENTION

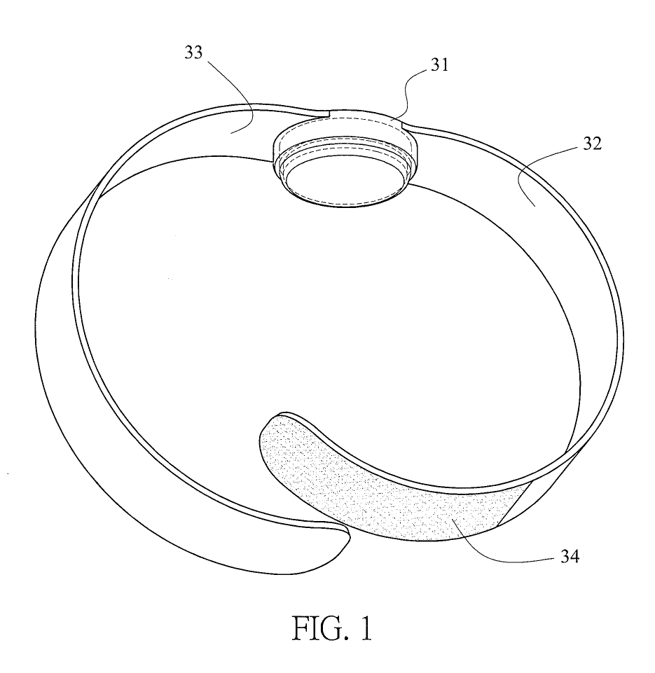

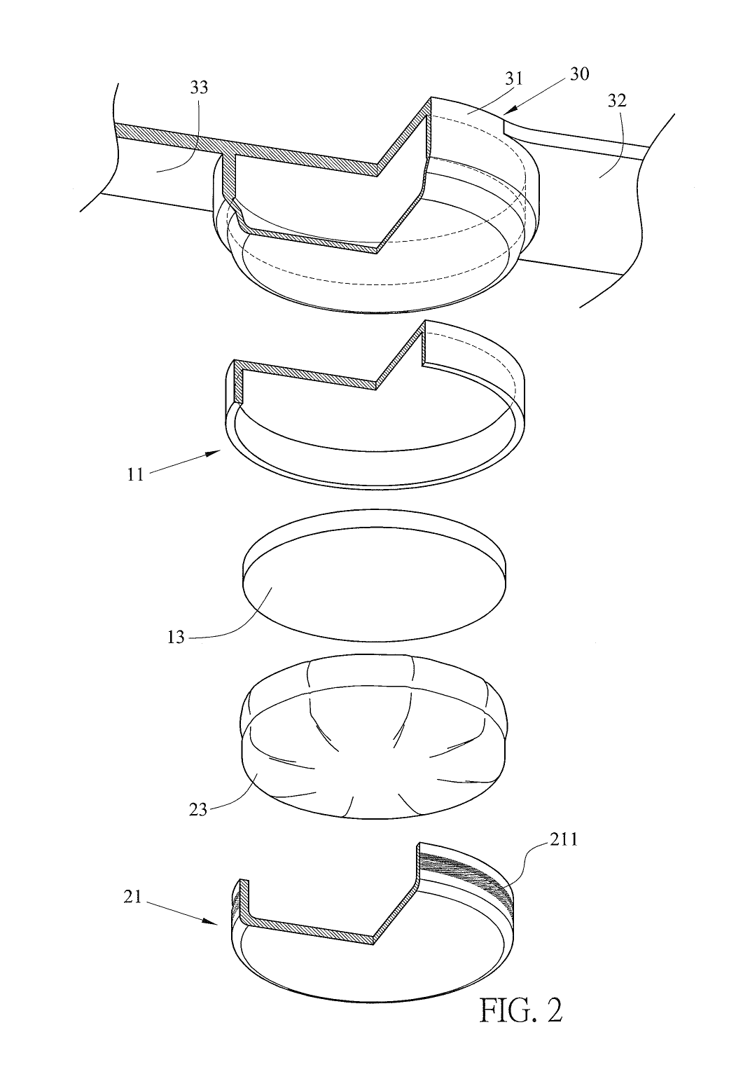

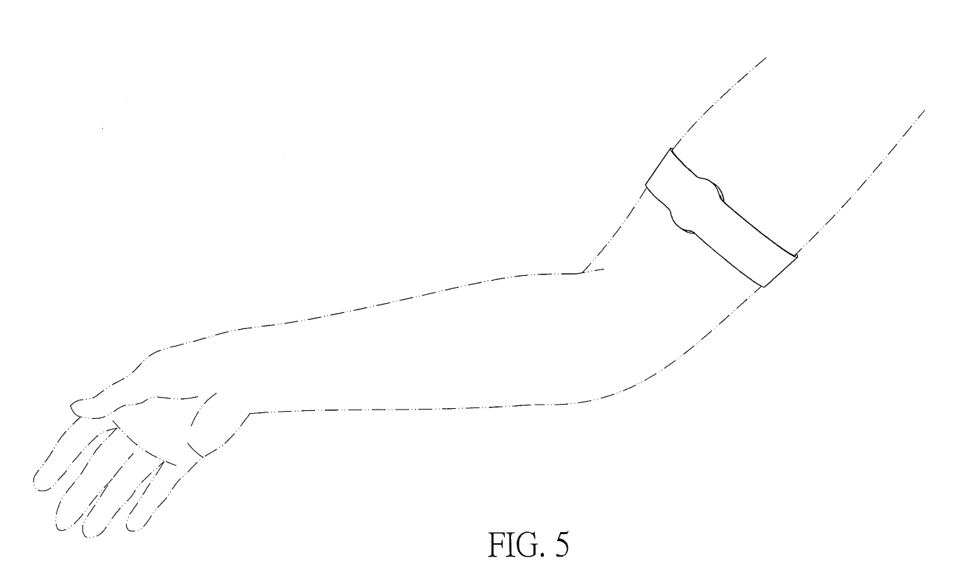

Referring to FIGS. 1 to 5, an adhesive bandage in accordance with a first preferred embodiment of the invention comprises the following components as discussed in detail below.

A sliding cup 11 is opaque and comprises a space 12 defined therein. A sliding disc 13 is fitted in the space 12. The sliding disc 13 comprises an activation member 131 on one surface and a guide member 132 on a periphery.

A support cup 21, opposite to the sliding cup 11, comprises a space 22 defined therein. A circular cushion member 23 is fitted in the space 22. The cushion member 23 is capable of resisting pressure of at least 200 mmHg. The support cup 21 is partially disposed in the sliding cup 11 with the cushion member 23 compressed between the support cup 21 and the stationary activation member 131. An annular marking 211 is formed on a periphery of the support cup 21. In one configuration, the marking 211 consists of a first ring, a second ring and a third ring with different colors adjacent to one another from top to bottom. Alternatively, in another configuration, the marking 211 consists of a scale. Alternatively, in still another configuration, the marking 211 is a combination of a first ring, a second ring and a third ring with different colors adjacent to one another from top to bottom, and a scale. Preferably, the first ring is a green ring, the second ring is a yellow ring and the third ring is a red ring.

A main body 30 is formed of silicone rubber and formed integrally. The main body 30 comprises a transparent dome 31 with the above components including the support cup 21, the cushion member 23, the sliding disc 13, and the sliding cup 11 concealed therein; a first adhesive strip 32 extending from the periphery of the dome 31; a second adhesive strip 33, opposite to the first adhesive strip 32, extending from the periphery of the dome 31; a hook and loop fabric fastener 34 formed on ends of the first and second adhesive strips 32 and 33; and an annular resilient section 35 formed on an intermediate portion of a periphery of the dome 31.

The hook and loop fabric fastener 34 can be releasably pressed together for easy fastening in order to affix the adhesive bandage to a cut or the like on the skin of a patient.

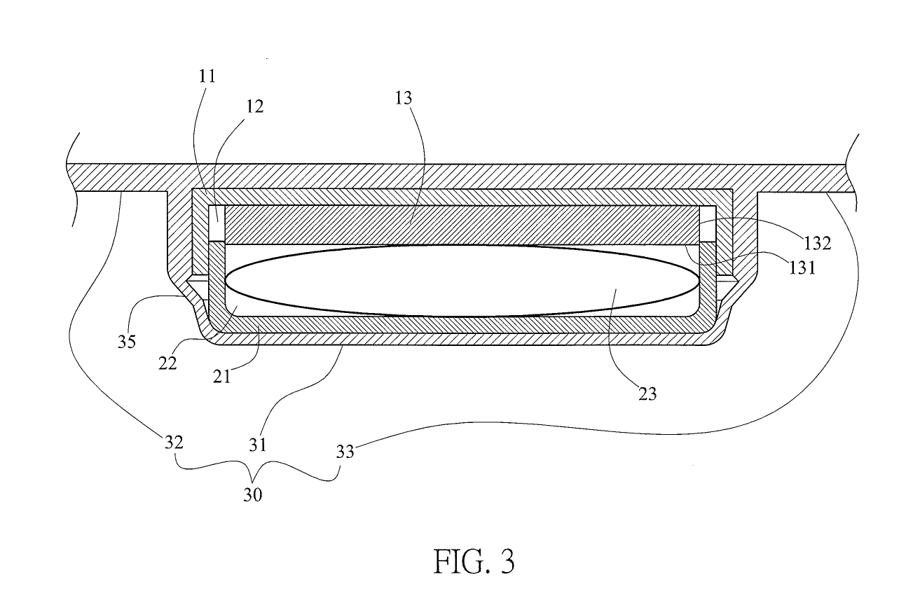

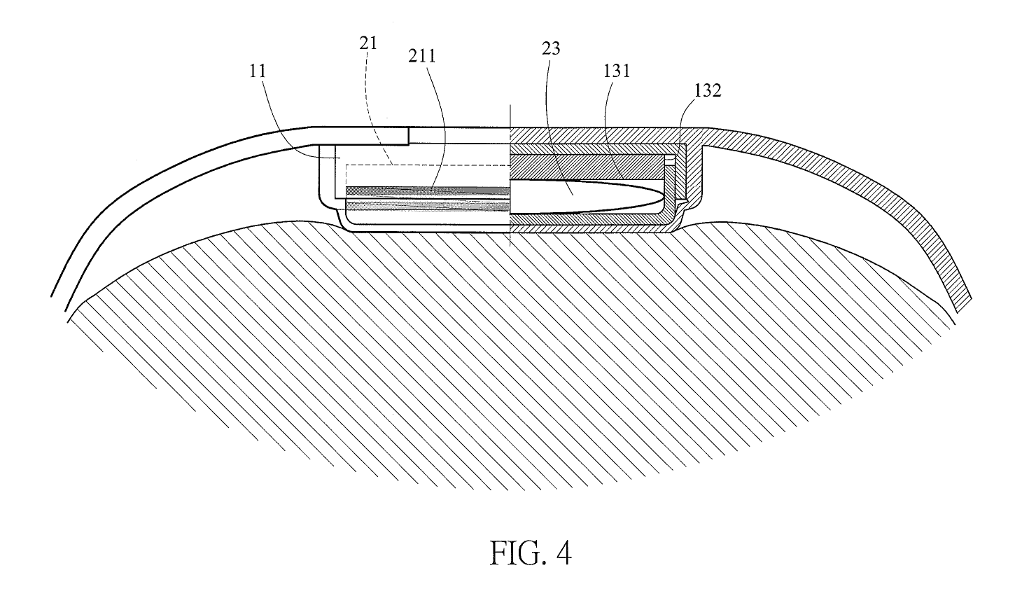

As shown in FIGS. 3 and 4, the sliding cup 11 slides in an annular gap between the support cup 21 and the periphery of the dome 31 toward the support cup 21, and the sliding disc 13 (i.e., the guide member 131) slides toward and into the space 22 by compressing the cushion member 23 when the annular resilient section 35 is compressed in response to pressing a joining portion of the dome 31, the first adhesive strip 32, and the second adhesive strip 33 by a medical employee. In detail, the cushion member 23 is compressed by the activation member 131 in response to a sliding movement of both the sliding cup 11 and the sliding disc 13 with respect to the support cup 21. The medical employee can clearly see the change of pressure applied to the adhesive bandage by observing the relative movement of a bottom edge of the sliding cup 11 and the marking 211(see FIG. 4).

The first ring (i.e., the green ring)of the marking 211 represents pressure applied to the adhesive bandage is less than 50 mmHg, the second ring (i.e., the yellow ring) thereof represents pressure applied to the adhesive bandage is in the range of 50 to 100 mmHg, and the third ring (i.e., the red ring) represent pressure applied to the adhesive bandage is in the range of 100 to 150 mmHg. Therefore, a medical employee may adjust pressure applied to the adhesive bandage (i.e., the dome 31) by observing a scale change among the red, yellow and green rings (i.e., tightening or loosening).

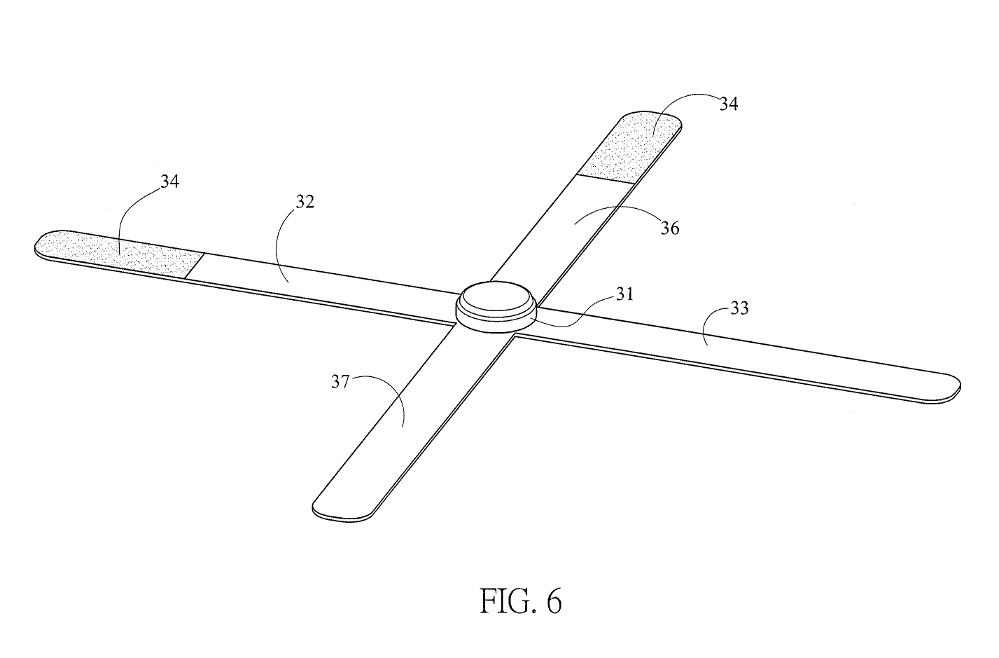

Referring to FIG. 6, an adhesive bandage in accordance with a second preferred embodiment of the invention is shown. The characteristics of the second preferred embodiment are substantially the same as that of the first preferred embodiment except the following:

A third adhesive strip 36 extends from the periphery of the dome 31; a fourth adhesive strip 37, opposite the third adhesive strip 36, extends from the periphery of the dome 31; and a hook and loop fabric fastener 34 formed on ends of the third and fourth adhesive strips 36 and 37. The first, third, second and fourth adhesive strips 32, 36, 33 and 37 are spaced from one another by 90-degree. Preferably, the adhesive bandage of the second preferred embodiment is adapted to apply to the joint for stopping bleeding.

While the invention has been described in terms of preferred embodiments, those skilled in the art will recognize that the invention can be practiced with modifications within the spirit and scope of the appended claims.

What is claimed is:

1. An adhesive bandage comprising:
a support cup comprising a space defined therein, and an annular marking formed on a periphery of an outer surface;
a circular cushion member fitted in the space of the support cup;
a sliding cup comprising a space defined therein;
a sliding disc disposed in the space of the sliding cup and engaged the cushion member, the sliding disc being capable of sliding into the space of the support cup and including an activation member formed on one surface and a guide member formed on a periphery; and
a main body formed of silicone rubber and comprising a transparent dome for concealing the sliding cup, the cushion member, the sliding disc, and the support cup, a first adhesive strip extending from a periphery of the dome; a second adhesive strip, opposite the first adhesive strip, extending from the periphery of the dome; a hook and loop fabric fastener formed on ends of the first and second adhesive strips wherein the hook and loop fabric fastener is capable of being pressed together for releasable fastening;
wherein the sliding disc compresses the cushion member in response to a sliding movement of the sliding cup with respect to the support cup so that a change of pressure applied to the adhesive bandage is visually brought about by a relative movement of a bottom edge of the sliding cup and the marking.

2. The adhesive bandage of claim 1, wherein the marking comprises a first ring, a second ring and a third ring with different colors adjacent to one another from top to bottom.

3. The adhesive bandage of claim 1, wherein the marking comprises a scale member.

4. The adhesive bandage of claim 1, wherein the marking comprises a combination of a first ring, a second ring, and a third ring with different colors adjacent to one another, and a scale member.

5. The adhesive bandage of claim 1, wherein the main body is formed integrally.

6. The adhesive bandage of claim 1, wherein the main body further comprises an annular resilient section formed on an intermediate portion of a periphery of the dome for allowing the sliding movement of the sliding cup.

7. The adhesive bandage of claim 2, wherein the first ring of the marking represents pressure applied to the adhesive bandage is less than 50 mmHg, the second ring thereof represents pressure applied to the adhesive bandage is in the range of 50 to 100 mmHg, and the third ring represents pressure applied to the adhesive bandage is in the range of 100 to 150 mmHg.

8. The adhesive bandage of claim 4, wherein the first ring of the marking represents pressure applied to the adhesive bandage is less than 50 mmHg, the second ring thereof represents pressure applied to the adhesive bandage is in the range of 50 to 100 mmHg, and the third ring represents pressure applied to the adhesive bandage is in the range of 100 to 150 mmHg.

9. The adhesive bandage of claim 1, further comprising a third adhesive strip extending from the periphery of the dome, a fourth adhesive strip extending from the periphery of the dome, and a hook and loop fabric fastener formed on ends of the third and fourth adhesive strips, and wherein the first, third, second and fourth adhesive strips are spaced from one another by 90-degree.
